(19) United States
(12) Patent Application Publication
(10) Pub. No.: US 2022/0401472 A1
(43) Pub. Date: Dec. 22, 2022

(54) COMPOSITIONS FOR PREVENTION AND TREATMENT OF RSV AND CORONAVIRUS INFECTION

(71) Applicant: Frank Joseph Reed, III, Garnet Valley, PA (US)

(72) Inventor: Frank Joseph Reed, III, Garnet Valley, PA (US)

(21) Appl. No.: 17/657,508

(22) Filed: Mar. 31, 2022

Related U.S. Application Data
(63) Continuation of application No. PCT/US2021/043903, filed on Jul. 30, 2021.
(60) Provisional application No. 63/060,358, filed on Aug. 3, 2020.

(51) Int. Cl. A61K 33/38; A61K 9/48; A61K 47/22; A61K 9/00; A61P 31/14

(52) U.S. Cl. CPC A61K 33/38; A61K 9/4891; A61K 47/22; A61K 9/0043; A61P 31/14

(57) ABSTRACT

Compositions and methods are provided for the prevention or treatment of RSV infection and SARS-CoV2 virus related infections, such as COVID-19 (2019-nCoV), in a human. The methods include administering one or more doses of a composition comprising an encapsulated nano-metal oxide (NMO) or nano-metal oxide chelate (NMO-Ch). The dose can be formulated for topical or parenteral administration. Topical administration includes administration as a nasal spray, or by inhalation of respirable particles or droplets. Particles or droplets contain material systems that include composite particles having a core and one or more shells that enclose the core. In this case, the shell is a nonlamellar amorphous material, and the internal matrix core contains a metal oxide or metal oxide chelate.

### PRIORITY

This application is a continuation of and claims priority to PCT International Application PCT/US2021/043903 filed Jul. 30, 2021, which is a PCT international filing of and claims priority from Provisional Application No. 63/060,358 filed Aug. 3, 2020, all entitled “Compositions for Prevention and Treatment of RSV and Coronavirus Infection”. All above provided applications are hereby incorporated by reference in their entirety.

### BACKGROUND

Since its discovery more than 60 years ago respiratory syncytial virus (RSV) has been recognized as the leading cause of lower respiratory tract illness (LRTI) in infants and young children worldwide. RSV, which is a member of the family Pneumoviridae, has a genome comprised of non-segmented negative-sense RNA that encodes eleven viral proteins. Infection with RSV typically begins in the epithelium of the upper respiratory tract and rapidly descends toward lower airways by intracellular transmission. It is responsible for a variety of symptoms ranging from low-grade fevers to severe bronchiolitis or pneumonia. On average, there are 33.1 million new infections each year, with 3.2 million hospital admissions, and 59,600 in-hospital deaths in children less than five years of age. Currently, Palivizumab, a monoclonal antibody, is the only prophylactic medication approved for use in infants. To date, there are no known effective vaccines or specific treatment options for RSV infections.

In late 2019, a novel coronavirus was identified as the cause of a cluster of pneumonia cases in Wuhan, a city in China. It subsequently spread throughout China and elsewhere, becoming a global health emergency. In February 2020, the World Health Organization designated the disease COVID-19, which stands for coronavirus disease 2019. Previously, this virus was referred to as 2019-nCoV.

Coronaviruses are classified as a family within the Nidovirales order, viruses that replicate using a nested set of mRNAs (“nido-” for “nest”). The coronavirus subfamily is further classified into four genera: alpha, beta, gamma, and delta coronaviruses. The human coronaviruses (HCoVs) are in two of these genera: alpha coronaviruses (HCoV-229E and HCoV-NL63) and beta coronaviruses (HCoV-HKU1, HCoV-0C43, Middle East respiratory syndrome coronavirus [MERS-CoV], the severe acute respiratory syndrome coronavirus [SARS-CoV]), and SARSCoV-2. SARS-CoV-2 is the cause of COVID-19. (Centers for Disease Control and Prevention. Human Coronavirus Types. www.cdc.gov/coronavirus/types.html).

Coronaviruses are medium-sized enveloped positive-stranded RNA viruses and the name derives from their characteristic crown-like appearance in electron micrographs. These viruses have the largest known viral RNA genomes, with a length of 27 to 32 kb (1 kb=300 nm so 8100-9600 nm). The host-derived membrane is studded with glycoprotein spikes and surrounds the genome, which is encased in a nucleocapsid that is helical in its relaxed form but assumes a roughly spherical shape in the virus particle. Replication of viral RNA occurs in the host cytoplasm by a unique mechanism in which RNA polymerase binds to a leader sequence and then detaches and reattaches at multiple locations, allowing for the production of a nested set of mRNA molecules with common 3′ ends.

Nanomedicine is a fast-growing field that utilizes nanotechnology to enhance applications, such as pharmaceuticals, diagnostic devices, and drug delivery systems. Nanomaterials range in size from 1-100 nm and are classified according to their size, shape, and biological interactions. Silver nanoparticles (AgNPs), defined as a cluster of colloidal silver, have been utilized in experimental models of infectious diseases as engineered AgNPs display the antimicrobial properties of bulk silver, with a significant reduction in the toxic effects observed with silver ions. The antimicrobial effects of AgNPs are accomplished by a unique physiochemical property which allows for a large surface area to volume ratio, generating more efficient contact with microorganisms and enhancing interactions with microbial proteins. This has garnered much success on an antibacterial platform, allowing for potential alternatives to antibiotic-resistant strains of bacteria, improved wound healing, and antibacterial coatings for medical materials, such as stents, catheters and orthopedic implants. AgNPs have also demonstrated promising antiviral capabilities with viruses, such as HIV, Tacaraibe virus, and several respiratory pathogens, including adenovirus, parainfluenza and influenza (H3N2).

By virtue of its natural function, the respiratory tract is exposed to a slew of airborne pathogens that cause a variety of respiratory ailments. Viral infection of the respiratory tract is the most common cause of infantile hospitalization in the developed world with an estimated 91,000 annual admissions in the US at a cost of $300 M. Human respiratory syncytial virus (RSV) and parainfluenza virus (PIV) are two major agents of respiratory illness; together, they infect the upper and lower respiratory tracts, leading to croup, pneumonia and bronchiolitis (Openshaw, P. J. M. Respir. Res. 3 (Suppl 1), S15-S20 (2002), Easton, A. J., et al., Clin. Microbiol. Rev. 17, 390-412 (2004)).

RSV alone infects up to 65% of all babies within the first year of life, and essentially all within the first 2 years. It is a significant cause of morbidity and mortality in the elderly as well. Immunity after RSV infection is neither complete nor lasting, and therefore, repeated infections occur in all age groups. Infants experiencing RSV bronchiolitis are more likely to develop wheezing and asthma later in life. Research for effective treatment and vaccine against RSV has been ongoing for nearly four decades with few successes (Openshaw, P. J. M. Respir. Res. 3 (Suppl 1), S15-S20 (2002), Maggon, K. et al, Rev. Med. Virol. 14, 149-168 (2004)).

Currently, no vaccine is clinically approved for RSV. Strains of RSV also exist for nonhuman animals such as the cattle, goat, pig and sheep, causing loss to agriculture and the dairy and meat industry (Easton, A. J., et al., Clin. Microbiol. Rev. 17, 390-412 (2004)).

Both RSV and PIV contain nonsegmented negative-strand RNA genomes and belong to the Paramyxoviridae family. A number of features of these viruses have contributed to the difficulties of prevention and therapy. The viral genomes mutate at a high rate due to the lack of a replicational proof-reading mechanism of the RNA genomes, presenting a significant challenge in designing a reliable vaccine or antiviral (Sullender, W. M. Clin. Microbiol. Rev. 13, 1-15 (2000)). Promising inhibitors of the RSV fusion protein (F) were abandoned partly because the virus developed resistant mutations that were mapped to the F gene (Razinkov, V., et. al., Antivir. Res. 55, 189-200 (2002), Morton, C. J. et al. Virology 311, 275-288 (2003)). Both viruses associate with cellular proteins, adding to the difficulty of obtaining cell-free viral material for vaccination (Burke, E., et al., Virology 252, 137-148 (1998), Burke, E., et al., J. Virol. 74, 669-675 (2000), Gupta, S., et al., J. Virol. 72, 2655-2662 (1998)). Finally, the immunology of both, and especially that of RSV, is exquisitely complex (Peebles, R. S., Jr., et al., Viral. Immunol. 16, 25-34 (2003), Haynes, L. M., et al., J. Virol. 77, 9831-9844 (2003)). Use of denatured RSV proteins as vaccines leads to “immunopotentiation” or vaccine-enhanced disease (Polack, F. P. et al. J. Exp. Med. 196, 859-865 (2002)). The overall problem is underscored by the recent closure of a number of anti-RSV biopharma programs.

The RSV genome comprises a single strand of negative sense RNA that is 15,222 nucleotides in length and yields eleven major proteins. (Falsey, A. R., and E. E. Walsh, 2000, Clinical Microbiological Reviews 13:371-84.) Two of these proteins, the F (fusion) and G (attachment) glycoproteins, are the major surface proteins and the most important for inducing protective immunity. The SH (small hydrophobic) protein, the M (matrix) protein, and the M2 (22 kDa) protein are associated with the viral envelope but do not induce a protective immune response. The N (major nucleocapsid associated protein), P (phosphoprotein), and L (major polymerase protein) proteins are found associated with virion RNA. The two non-structural proteins, NS1 and NS2, presumably participate in host-virus interaction but are not present in infectious virions.

Human RSV strains have been classified into two major groups, A and B. The G glycoprotein has been shown to be the most divergent among RSV proteins. Variability of the RSV G glycoprotein between and within the two RSV groups is believed to be important to the ability of RSV to cause yearly outbreaks of disease. The G glycoprotein comprises 289-299 amino acids (depending on RSV strain), and has an intracellular, transmembrane, and highly glycosylated stalk structure of 90 kDa, as well as heparin-binding domains. The glycoprotein exists in secreted and membrane-bound forms.

Successful methods of treating RSV infection are currently unavailable (Maggon K and S. Barik, 2004, Reviews in Medical Virology 14:149-168). Infection of the lower respiratory tract with RSV is a self-limiting condition in most cases. No definitive guidelines or criteria exist on how to treat or when to admit or discharge infants and children with the disease. Hypoxia, which can occur in association with RSV infection, can be treated with oxygen via a nasal cannula. Mechanical ventilation for children with respiratory failure, shock, or recurrent apnea can lower mortality. Some physicians prescribe steroids. However, several studies have shown that steroid therapy does not affect the clinical course of infants and children admitted to the hospital with bronchiolitis. Thus corticosteroids, alone or in combination with bronchodilators, may be useless in the management of bronchiolitis in otherwise healthy unventilated patients. In infants and children with underlying cardiopulmonary diseases, such as bronchopulmonary dysphasia and asthma, steroids have also been used.

In developed countries, the treatment of RSV lower-respiratory-tract infection is generally limited to symptomatic therapy. Antiviral therapy is usually limited to life-threatening situations due to its high cost and to the lack of consensus on efficacy. In developing countries, oxygen is the main therapy (when available), and the only way to lower mortality is through prevention.

The similarities between RSV and SARS-CoV2 virus related infections, such as COVID-19 (2019-nCoV), are numerous, including the way the virus spreads, symptoms that include runny nose, cough, fever, loss of appetite, trouble breathing, especially affecting persons with immune compromised systems and those over age 65. Both RSV and SARS-CoV-2 related infections possess an RNA genome, replicate in the cytosol of respiratory epithelial cells, provide self-capping structures for their mRNAs, and are found to create “replication organelles (ROs)” or other cystolic occluded structures.

Nano-silver colloids (NAgC) suppression of both viral and bacterial respiratory infections, formulations and treatment protocols based on NAgC by inhalation delivery have been presented with special evaluation given to coronavirus. Such NAgCs may be most effectively applied as a first-line intervention at an early stage of respiratory infections, i.e., when mostly affecting the upper respiratory system and bronchial tree. Therefore, the formulations could be used to control local outbreaks of COVID-19 via early stage home treatment. It is also noted that similar NAgC dosages also provide anti-bacterial effectiveness. For hospital ventilator associated pneumonia (VAP), inhalation delivery of NAgC can be implemented prophylactically to lower VAP risk. A recent publication provides data that indicates colloidal silver with particle sizes between 3 nm and 7 nm can be highly effective to suppress viral mechanisms of infection, and further concludes that IC50 concentrations of such colloids is about 10 μg/mL. (Zachar, O., ResearchGate.com, Preprint (2020), doi: 10.31219/osf.io/adnyb)

Several investigations reveal that some types of AgNPs (silver nano-particles) have been used for both in vitro and in vivo inhibition of fungus, bacteria, and viruses. Especially, in some reports, researchers used this nano-scale material for inhibition of corona virus family like H1V1, and H3N2 influenzas. It is suggested that at least the potential of silver nano particles as well as many non-hazard nano metals and nano metal oxides (by special dosages) could be considered as candidates for inhibition of 2019-nCoV. (Siadati, S., et al., Chem Rev Lett 3 (2020) 9-11).

One of the first in vivo animal studies in NAgC conducted on Influenza H3N2 infected mice (Xiang, D., et al. International Journal of Nanomedicine 2013:8 4103-4114) compares the effectiveness of intranasal administration of NAgC to that of Tamiflu (Oseltamivir). As shown in FIG. 1, NAgC is potentially as effective as Tamiflu and protected mice from H3N2 infection. FIG. 1 provides (A) survival rate changes (% and (B) Changes in body weight (%). Therefore, a NAgC treatment as effective as in the mice model, could cost less than 1/10 that of Tamiflu, could be available as an OTC option, and easily manufactured locally. While CoVs, rhinoviruses, and RSV replicate in the cytosol of respiratory epithelial cells and shield their replicating RNAs, influenza virus apparently takes another route, and as the only known exception to the rule this RNA virus replicates in the nucleus. (Kikkert, M., J Innate Immun 2020; 12:4-20)

Polyvinylpyrrolidone (PVP) is a linear polymer of 1-vinyl-2-pyrrolidone monomers used as a binder, emulsion stabilizer, film former, hair fixative, and suspending agent-nonsurfactant. The molecular weight of the polymer ranges from 10,000 to 700,000. PVP K-30, with an average molecular weight of 40,000, is typically used in cosmetic formulations. The highest concentration reported to be used is 35%. There was no significant absorption of PVP K-30 given orally to rats, and the acute oral LD₅₀ was >100 g/kg for rats and guinea pigs. Neither toxic effects nor gross lesions were found in rats maintained for two years on a diet containing 10% PVP K-30. Short-term PVP inhalation studies produced mild lymphoid hyperplasia and fibroplasia in rats, but no inflammatory response. (Final Report on the Safety Assessment of Polyvinylpyrrolidone (PVP). (International Journal of Toxicology, 17 (Suppl. 4):95-130, 1998)

U.S. Pat. No. 9,675,953 to Oldenburg et al. describes encapsulated particles where the material composition, comprises a plurality of particles, wherein each particle comprises a core and a shell encapsulating the core, the shell comprising at least one atomic element not included in the core, wherein the cores have: a median maximum dimension that is less than 10 microns, and a median of at least one axial dimension that is in the range of 10 nm to 500 nm, and wherein the shells have: a median thickness that is less than 100 nm, a silicon concentration that is in the range of 10% to 50% on the basis of the weight of the shells, and an aluminum concentration that is in the range of 0.01% to 5% on the basis of the weight of the shells, wherein the shells have a ratio of the aluminum concentration to the silicon concentration in the range of 1:20 to 1:5000 such that the median thickness does not change by more than 10% when measured 24 hours after immersing in water. The nanoparticles can be silver nanoparticles of spherical shape, and can be inorganic oxides of the selected metal. PVP-capped silver was treated to produce silicon oxide-shelled silver nanoparticles.

In US Pat. Appl. US2014/120168 to Oldenburg et al., PVP-capped silver was treated to produce silica-shelled silver nanoparticles.

US Pat. Appl. US2008/064767 to Chou, K. et al., describes the production of high-concentration silver colloidal solutions of mean particle size of less than 10 nm, and 20 nm or less for at least 180 days at room temperature, wherein the stabilizing agent can be selected from a group consisting of sodium dodecyl sulphate (SDS), polyvinyl pyrrolidone (PVP) and polyvinyl alcohol (PVA), and where the nanoscale silver composition comprises 1-5% w/w of metallic silver.

Microspherical particles of solid dispersions of polyvinylpyrrolidones K29-32 with model hydrophobic drug, PHE produced using spray drying, were studied using several physicochemical methods: SEM, DSC, PXRD, and FTIR. It is found that the spray drying process parameters enable producing particles with relatively low solvent content, on which

PHE stays in amorphous form for no less than 4 months. Particles prepared using spray caps with 7.0 μm hole size have optimal size distribution for inhalation administration. The drug content in microparticles was found to be excellent and was approximately close to the theoretical values. Measured dissolution kinetics of PHE evidence that dissolution rate of PHE from microspherical particles of solid dispersions allows their application as inhalation delivery systems with fast maximum concentration achievement time. (Urandar, S., et al., Ther. Deliv. (2018) 9(9), 667-689).

Chemical synthesis of AgNPs from AgNO₃ and Ethylene glycol with PVP stabilization produces silver nanospheres of 17±2 nm. A review of AgNPs provides that antiviral effects are achieved using PVP stabilization for: HIV-1, where the AgNps undergo a size-dependent interaction with HIV-1 (1-10 nm), and inhibit the virus from binding to the host; RSV, where PVP coated AgNPs effect 44% inhibition of RSV. (Tran, Q., et al., 2013 Adv. Nat. Sci: Nanosci. Nanotechnol. 4 (2013) 033001 (20 pp))

To evaluate possible adverse health effects, especially to the lungs, research was conducted on the cytotoxic and proinflammatory effects of AgNP after intratracheal instillation in rats. Monodisperse, polyvinylpyrrolidone (PVP)-coated AgNP (70 nm) showing little agglomeration in aqueous suspension were instilled intratracheally. After 24 hours, the lungs were lavaged, and lactate dehydrogenase (LDH), total protein, and cytokine levels as well as total and differential cell counts were measured in the bronchoalveolar lavage fluid (BALF). Instillation of 50 μg PVP-AgNP did not result in elevated LDH, total protein, or cytokine levels in BALF compared to the control, whereas instillation of 250 μg PVP-AgNP caused a significant increase in LDH (1.9-fold) and total protein (1.3-fold) levels as well as in neutrophil numbers (60-fold) of BALF. Furthermore, while there was no change in BALF cytokine levels after the instillation of 50 μg PVP-AgNP, instillation of 250 μg PVP-AgNP resulted in significantly increased levels of seven out of eleven measured cytokines. These finding suggest that exposure to inhaled AgNP can induce moderate pulmonary toxicity, but only at rather high concentrations. (Haberl, N. et al., Beilstein J. Nanotechnol. 2013, 4, 933-940).

In a further study, PVP-capped silver nanoparticles with a diameter of the metallic core of 70 nm, a hydrodynamic diameter of 120 nm and a zeta potential of −20 mV were prepared and investigated with regard to their biological activity. While primary brain astrocytes were shown to be fairly tolerant toward silver nanoparticles, silver nanoparticles induce the formation of DNA (double-strand-breaks (DSB) and lead to chromosomal aberrations and sister-chromatid exchanges in Chinese hamster fibroblast cell lines (CHO9, K1, V79B). An exposure of rats to silver nanoparticles in vivo induced a moderate pulmonary toxicity, however, only at rather high concentrations. The same was found in precision-cut lung slices of rats in which silver nanoparticles remained mainly at the tissue surface. In a human 3D triple-cell culture model consisting of three cell types (alveolar epithelial cells, macrophages, and dendritic cells), adverse effects were also only found at high silver concentrations. The silver ions that are released from silver nanoparticles may be harmful to skin with disrupted barrier (e.g., wounds) and induce oxidative stress in skin cells (HaCaT). In general, it can be said that only higher concentrations of silver nanoparticles induced biological responses in single cell cultures, in more complex systems and in animal experiments. Only for the fibroblast cultures, a genotoxicity was observed at rather low concentrations. It was determined that further coordinated efforts will be needed to explore the possible effects that silver nanoparticles can induce in (human) cells. (Ahlberg, S., et al., Beilstein J. Nanotechnol. 2014, 5, 1944-1965).

Colloidal synthesis offers a route to nanoparticles (NPs) with controlled composition and structural features. Polyvinylpyrrolidone (PVP) can be used to obtain such nanostructures. PVP can serve as a growth modifier, nanoparticle dispersant, reducing agent, and is a versatile shape-directing and stabilizing agent for monodisperse Ag nanostructure syntheses and is instrumental toward achieving shape control in Ag systems, with a variety of convex structures demonstrated. The role of PVP depends on the synthetic conditions. This dependence arises from the amphiphilic nature of PVP along with the molecular weight of the selected PVP. These characteristics can affect nanoparticle growth and morphology by providing solubility in diverse solvents, selective surface stabilization, and even access to kinetically controlled growth conditions. Empirically, PVP appears to stabilize {100} Ag facets in polyols based on the preference for {100}-encased structures achieved in its presence. Both experimental and theoretical work suggest that PVP lies flat on Ag surfaces and the higher binding energy of PVP to Ag {100} facets arises from van der Waals (vdW) attraction and direct binding through the oxygen atom. (Koczkur, K., et al., Dalton Transactions, Royal Society of Chemistry, 2015, 44 (41), pp. 17883-17905).

There have been recent developments in the field of drug delivery, medical therapeutics and diagnostics specifically involving the nonlamellar liquid crystalline (NLC) systems. Different NLC phases were highlighted having cubic, hexagonal and sponge internal structures, and their application in the field of drug delivery, such as dose reduction, toxicity reduction and therapeutic efficacy enhancement either in the form of nanoparticles, colloidal dispersion or gels. NLC systems could be designed to give extended drug-release profiles and impedes the rapid in vivo biodegradation of the sensitive pharmaceutical active agents. NLC colloidal nanoparticles are resistant to phase transitions even in excess hydration condition. (Urandar, S., et al., Ther. Deliv. (2018) 9(9), 667-689).

Previous studies have shown that the particle size required for ideal drug aerosol preparation is between 1 and 3 μm, and that the application of microspherical particles of solid dispersions enhances bioavailability of poorly soluble drugs due to the solubilization. In the present work, the spray drying process of the production of microspherical particles of solid dispersions of polyvinylpyrrolidone K29-32 with model hydrophobic drug, phenacetin, was optimized using the results of DSC, PXRD, and viscometry. The diameter of the obtained particles is within 1-3 μm range. The Gibbs energy of dissolution in water was shown to be negative for the mixture with polymer/phenacetin mass ratio 5:1. It was demonstrated that the optimal size distribution for the inhalation administration is obtained for microspherical particles produced using spray caps with 7.0 μm hole size. The dissolution rates of phenacetin from the produced microspherical particles were faster than that of drug powder. As evidenced by powder X-ray diffraction data, phenacetin remained in an amorphous state for 4 months in microspherical particles of solid dispersions. (Usmanova, L., et al., Bio Med Research International. Volume 2018, Article ID 2412156, 12 Pages).

Therefore a need for an amorphous, non-crystalline, system for respiratory drug-delivery exists, where in the pharmaceutical industry the amorphous drugs were shown to have higher bio-availability than their crystalline counterparts due to the high solubility of the amorphous phase.

Recent studies, undertaken on behalf of the inventor and disclosed herein, have identified the broad-spectrum antiviral properties of silver nanoparticles (AgNPs) effectiveness against respiratory pathogens, such as adenovirus, parainfluenza, and influenza. AgNPs achieve this by attaching to viral glycoproteins, blocking entry into the host cell. An objective of the present disclosure is based upon a recent study performed to evaluate the antiviral and immunomodulatory effects of AgNPs in RSV infection. AgNP-mediated reduction in RSV replication was demonstrated, both in epithelial cell lines and in experimentally infected BALB/c mice. Marked reduction in pro-inflammatory cytokines (i.e., IL-1α, IL-6, TNF-β) and pro-inflammatory chemokines (i.e., CCL2, CCL3, CCL5) was also observed. Conversely, CXCL1, G-CSF, and GM-CSF were increased in RSV-infected mice treated with AgNPs, consistent with an increase of neutrophil recruitment and activation in the lung tissue. Following experimental antibody-dependent depletion of neutrophils, the antiviral effect of AgNPs in mice treated was ablated. This is the first in vivo report demonstrating antiviral activity of AgNPs during RSV infection. (Morris, D., Viruses 2019, 11, 732; doi: 10.3390/v11080732).

Therefore, there is a need for safe and effective vaccines against RSV and SARS-CoV2 virus related infections, such as COVID-19 (2019-nCoV), especially for infants and children, immune-compromised, and persons over 65 years of age. There is also a need for therapeutic agents and methods for treating RSV infection and SARS-CoV2 virus related infections, such as COVID-19 (2019-nCoV), at all ages and in immuno-compromised individuals. There is also a need for scientific methods to characterize the protective immune response to RSV and SARS-CoV2 virus related infections, such as COVID-19 (2019-nCoV), so that the pathogenesis of the disease can be studied, and screening for therapeutic agents and vaccines can be facilitated. The present disclosure overcomes previous shortcomings in the art by providing compositions and methods effective for modulating or preventing RSV infection and SARS-CoV2 virus related infections, such as COVID-19 (2019-nCoV). Specifically, the present disclosure advances the art by providing encapsulated metal oxide or chelated metal oxide agents; wherein, a metal oxide or chelated metal oxide particle coated with a nonlamellar amorphous material includes an internal matrix core having at least one nanostructured liquid phase, or a combination of the two used for the delivery of active agents including pharmaceuticals, nutrients, pesticides, etc. The coated particle can be fabricated by a variety of different techniques where the exterior coating is a nonlamellar amorphous material.

### SUMMARY

Many, if not all viruses, including respiratory viruses, suppress innate immune response to gain a window of opportunity for efficient virus replication and setting-up of infection. Common viruses that invade the lungs: coronaviruses (CoVs), rhinoviruses, RSV, and influenza, all possess an RNA genome.

Viruses with an RNA genome produce several RNA species during viral replication, which are normally absent in uninfected cells. For example, dsRNA and RNA with a 5′-triphosphate are commonly produced by RNA viruses during replication, but since the host cells do not normally copy RNA from RNA templates, these intermediate RNA species are recognized by innate immune sensors as foreign, resulting in antiviral effector activation. To be able to set up a productive infection in the cell, these viruses therefore need to circumvent and/or suppress these intracellular innate antiviral responses. An obvious primary strategy would be to shield away the replication intermediates with their dangerous, recognizable features, from the innate immune sensors roaming the cytosol. Indeed, the viruses that have a +RNA genome, which replicate exclusively in the cytosol such as the CoVs and rhinoviruses that invade the lungs, generally modify intracellular membranes elaborately to form headquarters of viral RNA replication, also called “replication organelles” (ROs; CoVs), “replication factories,” “double membrane vesicles” (DMVs; CoVs, enteroviruses), “invaginations,” or other. Also, the negative-stranded RSV genome and its replication enzymes are found associated with cytosolic occluded structures, in that case named inclusion bodies. Expression of a selection of specific hydrophobic viral proteins can usually mimic the formation of these structures, for example, nsp3 and nsp4 of CoVs, the N and P proteins of RSV, and 2B, 2C and 3A proteins of enterovirus (polio). All these structures, while diverse in morphology and contents, seem to concentrate the viral replication machinery, intermediates and products inside membrane-bound vesicles or invaginations, seemingly unreachable for the innate immune sensors of the cytosol.

As an antiviral agent, AgNPs have been proposed to interfere with viral replication by two separate mechanisms. The first is by binding via sulfur-bearing residues on surface glycoproteins, preventing the attachment and entry of the virus into the host cell. The second mechanism involves AgNPs crossing the cell membrane and effectively blocking cellular factors necessary for the proper assembly of viral progeny. The RSV virion contains two surface glycoproteins (F and G) that it utilizes to gain access to the host cell, where it replicates primarily within the cytoplasm. Therefore, AgNPs have the potential to block entry of RSV by binding of surface glycoproteins and/or inhibit the spread of RSV from within the host cell.

Based on the success of AgNPs with similar pathogens, the current study was designed to analyze the antiviral and immunomodulatory capacity of AgNPs against RSV infection, utilizing both in vitro and in vivo models. Significant reductions of RSV replication are shown in A549 and HEp-2 cell lines, as well as in experimentally infected BALB/c mice. Also demonstrated was a shift in the cytokine profile within the bronchoalveolar lavage fluid (BALF) of mice inoculated with AgNP-RSV to one that supports neutrophil recruitment and activation. Upon depletion of neutrophils with anti-Ly6G, the antiviral effects of AgNPs were reversed, suggesting neutrophils as a primary mechanism to the antiviral activity presented by AgNPs in vivo. Insight is given herein to the antiviral mechanisms of AgNPs and their potential therapeutic application for RSV infections, and further applications to SARS-CoV2 virus related infections, such as COVID-19 (2019-nCoV).

Inhalation therapy is an express method of drug delivery into the human circulatory system. Due to extremely abundant capillary network and enormous alveoli area drugs are absorbed extremely rapidly. The inhaled chemicals are not subjected to the bio-transformations, which occur in the digestive tract, and drug activity is not reduced in the liver. Drugs introduced into lungs via inhalation have 10 to 200 times higher bioavailability than by nasal or gastrointestinal administration. Furthermore, inhalation administration of drug compound in an aerosol with controlled particle size and shape may serve as a noninvasive alternative to injection of medicines.

In an inhalation therapy, inhaled drugs enter the body as aerosols. Therapeutically active particle size is limited between 1 and 5 μm. Particles with a diameter higher than 10 μm are deposited in the oropharynx, those measuring between 5 and 10 μm accumulate in the central airways, and those from 0.5 to 5 μm enter the small airways and alveoli. In order to target the alveolar region specifically, the aerosol particle diameter should not exceed 3 μm. Particles with a diameter below 1 μm are exhaled during normal tidal breathing. Moreover, the work demonstrated that particles less than 1 μm in size are more readily subjected to phagocytosis. Therefore, it has been previously determined that the ideal drug aerosol must have particles with a diameter in 1 to 3 μm range.

On the other hand, most novel drugs are poorly soluble in water limiting their bioavailability. The most convenient means for “hydrophilization” of drugs is solid dispersion formation.

Biocompatible polymers including polyvinylpyrrolidones (PVP) have found wide application as water-soluble matrix for drug delivery. It was earlier demonstrated that PVP produces solid dispersions with a wide range of compounds and that a decrease of cytotoxicity of active pharmaceutical ingredients (API) occurs due to solid dispersion formation. The fact that PVP shows protective properties towards macrophage action on compounds encapsulated in the polymer is shown. PVP is among most promising materials as its biocompatibility and its usage as plasma replacement illustrate the almost complete lack of toxicity.

Presently one of the main methods of nano- and microparticle preparation is the spray drying process, which has found a good use for commercial-scale production for inhaler products. This method enables the production of spherical particles with sharp size distribution, as well as solid dispersions of drugs. Furthermore spherical particles demonstrate lower toxic effects than that of other shapes, which makes spray drying the most promising method for the preparation of medical products for inhalation administration. Only a limited number of commercially available devices allow production of spherical particles of the 1-3 μm size range. The production rate of such devices is usually limited and strongly depends on the viscosity (concentration) of the feeder solution and temperature (feed rate) of the desiccant. The parameters of the spray drying process also have a significant effect on the morphology of produced particles.

More specifically, the present disclosure describes a composition for inhibiting respiratory viruses including RSV and SARS-CoV2 related infections comprising; encapsulated metal oxides or chelated metal oxide molecules wherein the metal oxide(s) molecules are silver oxide molecules in solution and wherein the silver oxide(s) molecules are present in a concentration of between 5 and 250 parts per million in the solution.

Here, the solvent provides a liquid solution of encapsulated metal oxides or chelated metal oxide molecules wherein the solvent is selected from one or more of a group consisting of water, alcohol, plant-derived glycerine, and aloe.

In a further embodiment, the silver oxide molecules are encapsulated with one or more biopolymers that have a core and one or more shells that enclose the core. The shells can be comprised of a nonlamellar amorphous material and wherein the core contains the silver oxide molecules.

In this case, the biopolymers are polyvinylpyrrolidone (PVP) as provided in Structure (1), or another water-soluble polymer of N-vinylpyrrolidone (NVP) or a derivative thereof as provided in structure (2).

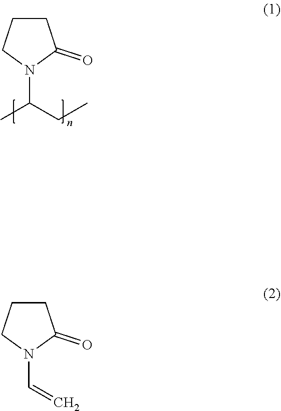

It is an object of the present disclosure to include silver oxide molecules encapsulated with the biopolymers which are either silver oxide or chelated silver oxide nanoparticles with a particle size of between 1 nm and 20 nm.

The silver oxide nanoparticles exhibit a high degree of inhibition of the viruses specifically in a range of 8 nm to 11 nm.

The silver oxide nanoparticles reduce RSV replication and production of pro-inflammatory cytokines in epithelial cell lines and in mouse lung that is mediated by neutrophils.

In addition to the composition described above, a method of inhibiting respiratory viruses includes RSV and SARS-CoV2 related infections is described that comprises inhalation and/or intranasal administration of doses of encapsulated metal oxide or chelated metal oxide molecules wherein the metal oxides are silver oxides and are provided in a liquid solution wherein a solvent provides a solution of encapsulated metal oxides or chelated metal oxide molecules wherein the solvent is selected from one or more of a group consisting of water, alcohol, plant-derived glycerine, and aloe.

The method includes parenteral administration of the molecules.

The method is useful in reducing RSV mRNA levels, RSV protein levels and RSV viral titers in a subject, the subject including a mammal, and a human.

Administration of multiple doses of encapsulated silver oxide or chelated silver oxide molecules over a course of days provides a therapeutically effective amount of chelated silver oxide.

A further embodiment includes the use of a first of a plurality of doses that is administered in a prophylactic manner before a subject is infected with RSV or a SARS-CoV2 virus related infection, such as COVID-19.

An additional risk reduction benefit of an inhalation treatment of an encapsulated metal oxide or chelated metal oxide composition for ventilated patients includes suppression of biofilm formation inside an endotracheal or tracheostomy tube.

In another embodiment, the liquid solution is formulated to have an osmolality ranging from 200-400 mOsm/kg.

In yet another embodiment, the liquid solution is buffered and a pH of the liquid solution is between 5 and 8.

The composition can be administered as an aerosolized liquid such as a nasal spray, wherein the aerosolized liquid is produced by a nebulizer.

It is possible to include a 0.1 ml to 0.6 ml of the aerosolized liquid comprising an encapsulated metal oxide or metal oxide chelate that is administered to each nostril.

In addition, it is possible for a plurality of doses to be administered daily, wherein a plurality includes two, three, four, or five doses.

Administering of the plurality of doses reduces RSV protein, mRNA, or titer in a cell of a human or mammalian respiratory tract to at least a level of administering a single dose that equals a dose provided by the plurality of doses.

Administering of the plurality of doses by inhalation delivers a total dose of between 0.1 and 0.6 mg/kg of an encapsulated metal oxide or metal oxide chelate to a mammal and/or human.

The present disclosure is also based on the in vitro and in vivo demonstration that respiratory viruses, such as RSV or a SARS-CoV2 virus related infection, such as COVID-19 (2019-nCoV) are inhibited through inhalation and/or intranasal administration of encapsulated metal oxide or chelated metal oxide agents, as well as by parenteral administration of such agents. Based on these findings, the present disclosure provides specific compositions and methods that are useful in reducing RSV mRNA levels, RSV protein levels and RSV viral titers in a subject, e.g., a mammal, such as a human. It is shown herein that administration of multiple doses of an encapsulated metal oxide or chelated metal oxide agent over a course of days can provide improved results.

In yet another aspect, the present disclosureprovides for an encapsulated chelated metal oxide composition that comprises a therapeutically effective amount of chelated silver oxide.

In another aspect, the metal oxide or chelated metal oxide is a nanoparticle, such as chelated nano-silver metal oxide of particle size 1 nm to 20 nm, preferably 8 nm to 11 nm.

In further aspect, the metal oxide or chelated metal oxide is present at a concentration of 5 ppm to 250 ppm, and preferably 100 ppm.

In one embodiment the present disclosureprovides for a lyophilized powder. In another embodiment the present disclosureprovides for a liquid solution, and in another embodiment a liquid suspension, and in another embodiment a dry powder comprising said amount of encapsulated metal oxide or metal oxide chelate.

In one related embodiment of the present disclosure a liquid solution is formulated to have an osmolality ranging from 200-400 mOsm/kg. In certain embodiments, the liquid solution is buffered. In certain embodiments, the pH of the liquid solution is between 5 and 8. In other embodiments, the pH of the liquid solution is between 5.6 and 7.6. In related embodiments, the liquid solution comprises a sodium phosphate buffer. In still other embodiments, the concentration of the buffer is between 10 and 100 mM, between 20 and 80 mM, between 30 and 70 mM, between 40 and 60 mM, or equal to or about 50 mM. In yet another embodiment, the pH of the buffered solution is 6.6.

In yet another related embodiment, a therapeutic encapsulated metal oxide or metal oxide chelate composition is administered topically. In related embodiments, the topical administration is intranasal or intrapulmonary, e.g., administration occurs by inhalation of said composition. In still other related embodiments, the patient administers the composition to himself or herself, or a third party (e.g., a guardian or a healthcare practitioner such as a doctor) can administer the composition to the patient. In certain embodiments, the composition is administered as an aerosolized liquid, e.g., a nasal spray. The nasal spray can be administered a Becton-Dickinson Accuspray™ nasal spray system or an equivalent thereof. In related embodiments, the aerosolized liquid is produced by a nebulizer. In still other related embodiments, 0.1 ml to 0.6 ml of the aerosolized liquid comprising an encapsulated metal oxide or metal oxide chelate is administered to each nostril. A plurality of doses can be administered daily, where a plurality includes two, three, four, or five doses. In related embodiments, the administering of the plurality of doses reduces RSV protein, mRNA, or titer in a cell of the respiratory tract of said human to at least the same level as an administering of a single dose that equals the dose provided by said plurality of doses. In yet other related embodiments, the administering of said plurality of doses by inhalation delivers a total dose of between 0.1 and 0.6 mg/kg of an encapsulated metal oxide or metal oxide chelate to said human.

In certain embodiments, the first of the plurality of doses is administered before the subject is infected with RSV or a SARS-CoV2 virus related infection, such as COVID-19 (2019-nCoV) (e.g., prophylactically).

In a further embodiment, an additional risk reduction benefit of an inhalation treatment of an encapsulated metal oxide or chelated metal oxide composition for ventilated patients is the possibility of suppression of biofilm formation inside the endotracheal or tracheostomy tube.

In still another embodiment, the disclosed technology generally relates to material systems which include a plurality of particles, and more particularly to material systems that include composite particles having a core and one or more shells that enclose the core, wherein the shell is a nonlamellar amorphous material, and the internal matrix core contains a metal oxide chelate.

In a another embodiment the encapsulant is polyvinylpyrrolidone (PVP) as provided in Structure (1), or another water-soluble polymer of N-vinylpyrrolidone (NVP) as provided in structure (2).

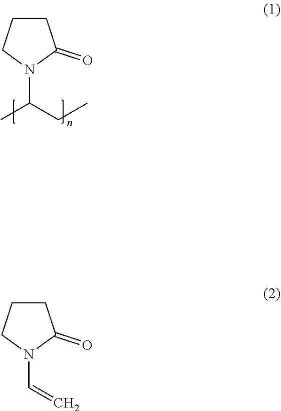

The details of one or more embodiments of the present disclosure are set forth in the accompanying drawings and the description below. Other features, objects, and advantages of the present disclosure will be apparent from this description, the drawings, and from the claims.

### BRIEF DESCRIPTION OF DRAWINGS

FIG. 1: In-vivo intranasal NAgC administration protected mice from H3N2 infection. (A) survival rate changes (%). (B) Changes in body weight (%). (Prior Art; Adapted from Xiang et al., “Inhibition of A/Human/Hubei/3/2005 (H3N2) influenza virus infection by silver nanoparticles in vitro and in vivo”. Int J Nanomedicine. 2013; 8(1):4103-4114, https://doi.org/10.2147/IJN.S53622).

FIG. 2A-2F: Graphical analysis of results showing silver nanoparticles (AgNPs) decreased RSV replication in epithelial lines.

FIG. 3A-3E: AgNPs decreased RSV replication in the lung tissue of experimentally infected mice.

FIG. 4A-4C: AgNPs decrease viral-induced cytokines, while increasing those associated with neutrophil recruitment and activation.

FIG. 5A-5F: AgNPs increase recruitment and activation of neutrophils to the lung, regardless of infection status.

FIG. 6A-6B: Neutrophil depletion results in a reversal of the antiviral effect noted in neutrophil immunocompetent AgNP-RSV mice.

### DETAILED DESCRIPTION

The instant specification may refer to one or more of the following abbreviations whose meanings are defined in Table 1, below.

TABLE 1: List of Abbreviations

A Adenosine

AgNPs Silver Nanoparticles

BAL Bronchoalveolar Lavage

BALF Bronchoalveolar Lavage Fluid

C Cytidine

CXCL1 Chemokine (C-X-C motif) Ligand 1

CXCL8 Chemokine (C-X-C motif) Ligand 8

Da Daltons

ELISA Enzyme-Linked Immunosorbent Assay

F12K Nutrient Mixture Ham's F-12K (Kaighn's Modification)

g Gram

H1N1 Influenza A virus subtype H1N1; A/H1N1

H3N2 Influenza A virus subtype H3N2; A/H3N2

HCV Hepatitis C Virus

HIV Human Immunodeficiency Virus

IFN Interferon

IL Interleukin

kg Kilogram

LDH Lactate Dehydrogenase

LRTI Lower Respiratory Tract Illness

M Molar

MEM Minimum Essential Media

mg Milligram

mL Milliliter

mM Millimolar

MOI Multiplicity of Infection

mRNA Messenger Ribonucleic Acid

NAgC Nano-silver Colloids

ND Not Detected

nm Nanometer

NT Not Tested

OTC Over the counter

p.i. post-infection

PBS Phosphate-Buffered Saline

pH Potential of Hydrogen

ppm Parts Per Million

PCR Polymerase Chain Reaction

qRT-PCR Quantitative Reverse Transcription PCR

RSV Respiratory Syncytial Virus

VAP Ventilator Acquired Pneumonia

vol/vol Volume per Volume

w/v Weight by Volume

w/w Weight by Weight

In the anti-viral uses of the present disclosure, silencing of a target gene will result in a reduction in “viral titer” in the cell or in the subject. As used herein, “reduction in viral titer” refers to a decrease in the number of viable virus produced by a cell or found in an organism undergoing the silencing of a viral target gene. Reduction in the cellular amount of virus produced will preferably lead to a decrease in the amount of measurable virus produced in the tissues of a subject undergoing treatment and a reduction in the severity of the symptoms of the viral infection.

As used herein, a “subject” refers to a mammalian organism undergoing treatment for a disorder mediated by viral expression, such as RSV infection or undergoing treatment prophylactically to prevent viral infection. The subject can be any mammal, such as a primate, cow, horse, mouse, rat, dog, pig, goat. In the preferred embodiment, the subject is a human.

As used herein, treating viral infection refers to the amelioration of any biological or pathological endpoints that 1) is mediated in part by the presence of the virus in the subject and 2) whose outcome can be affected by reducing the level of viral gene products present.

### Example

### Materials and Methods

### RSV Preparation

RSV Long strain was grown in HEp-2 cells and purified by centrifugation on discontinuous sucrose gradients. The titer of viral pools was determined by a methylcellulose plaque assay using HEp-2 cells, as described previously by Ueba (Ueba, O. Acta Med. Okayama 1978, 32, 265-272) and Kisch et al. (Proc. Soc. Exp. Biol. Med. 1963, 112, 583-589.). Virus pools were aliquoted, quick-frozen on dry ice-alcohol, and stored at −80 deg C. until needed.

### PVP-Coated Silver Nanoparticles (AgNP)

10 nm polyvinylpyrrolidone (PVP) coated BioPure™ silver nanospheres were purchased from NanoComposix Inc. (San Diego, Calif., USA). The PVP coating was chosen for its tight association with the silver particle, making the AgNP as stable as possible in a variety of different environments. Per the manufacturer, the AgNPs used in this study have a mass concentration of 1 mg/mL with a size distribution of 8-12 nm. The AgNPs exhibited an optimal density of 155 cm⁻¹ and a peak wavelength of 390 nm. Endotoxin concentrations were less than 5 EU/mL, and silver purity was 99.99%. TEM images of the AgNPs provided by the manufacturer are available upon request. For in vitro analyses, AgNPs were diluted in F12K or MEM (110 mM glutamine, 100 IU/mL penicillin, and 100 μg/mL streptomycin) to a total volume of 1 mL. For in vivo analyses, AgNPs were diluted in sterile phosphate-buffered saline (PBS) prior to mice inoculation.

### Studies In Vitro

A549 cells, a human alveolar type II-like epithelial cell line, and HEp-2 Cells (American Type Culture Collection, Manassas, Va., USA) were grown in F12K and MEM, respectively. Media contained 10% (vol/vol) FBS, 10 mM glutamine, 100 IU/mL penicillin, and 100 μg/mL streptomycin. Confluent monolayers were infected with RSV treated with varying doses of AgNPs (0, 10, 25 or 50 μg/mL). Samples were incubated with shaking for 1 h at room temperature prior to plating. A549 cells were infected at a multiplicity of infection (MOI) of 1 for 24 h. HEp-2 cells were infected at an MOI of 0.01 for 48 h. Supernatants were aliquoted and stored at −80 deg C. To evaluate viral titer, serial five-fold dilutions of infected supernatants were determined by plaque assay on HEp-2 cells under methylcellulose overlay. Plaques were visualized five days later, and viral titers were calculated as PFU/mL. Additionally, CXCL8 (IL-8) and CCL5 (RANTES) were also quantified by an enzyme-linked immunosorbent assay (ELISA) (R&D Systems, Minneapolis, Minn., USA).

The toxicity of AgNPs on epithelial cells was assessed in vitro with an A549 cell line, using lactate dehydrogenase (LDH) activity as an index of cellular damage. To measure LDH activity, A549 epithelial cells were exposed to varying doses of AgNPs (0, 10, and 50 μg/mL) for 24 h. LDH in the medium was measured by colorimetric assay using a commercially available kit (Cayman Chemical, Ann Arbor, Mich., USA) following the manufacturer's instructions. This assay measures cellular damage in response to chemical compounds or environmental factors using a coupled two-step reaction, as previously described.

A549 are adenocarcinomic human alveolar basal epithelial cells constituting a cell line that was first developed in 1972 by D. J. Giard, et al. through the removal and culturing of cancerous lung tissue in the explanted tumor of a 58-year-old Caucasian male. HEp-2 cells lines are from tumors which were produced in irradiated-cortisonised weanling rats after injection of epidermoid carcinoma tissue isolated from the larynx of a 56 year old male. STR (DNA)-profiling has revealed that the Hep-2 cell line is almost identical to the HeLa cell line.

### Viral Infection of Balb/c Mice

Female, 10 to 12-week-old BALB/c mice were purchased from Jackson Laboratory (Bar Harbor, Me., USA) and housed under pathogen-free conditions in the animal research facility of the University of Texas Medical Branch (UTMB), Galveston, Tex. BALB/c is an albino, laboratory-bred strain of the house mouse from which a number of common sub-strains are derived. Now over 200 generations from New York in 1920, BALB/c mice are distributed globally, and are among the most widely used inbred strains used in animal experimentation. All care and procedures involving mice in this study were in accordance with the recommendations in the Guide for the Care and Use of Laboratory Animals of the National Institutes of Health and UTMB institutional guidelines for animal care. A mixture of Ketamine (90-150 mg/kg) and Xylazine (7.5-16 mg/kg) was administered by intraperitoneal (IP) injection for anesthesia and euthanasia. This protocol was approved by the Institutional Animal Care and Use Committee of UTMB (protocol number 90010021).

The dosage of AgNPs was calculated based on the weight of the animals. All inoculants were incubated with shaking for 1 h at room temperature prior to inoculation. Under light anesthesia, mice were intranasally inoculated with 100 μL of sterile PBS as a mock inoculation, AgNPs (2 mg/kg or 4 mg/kg) diluted in PBS (denoted as AgNP-PBS), RSV diluted in PBS at a dose of 5×10⁶ PFU (denoted as RSV), or RSV mixed with AgNPs (2 mg/kg or 4 mg/kg) diluted in PBS (denoted as AgNP-RSV). Animals from all groups were evaluated on a daily basis for weight loss, illness score, and presence of respiratory symptoms. The percentage of bodyweight change was plotted over time. Clinical illness scores were visually determined by two investigators using a standardized 0-5 grading system (0-no disease, 1-slightly ruffed fur, 2-full ruffed fur, 3-ruffed fur and hunched back, 4-ruffed fur, hunched back and inactive, and 5-death). These parameters have been shown to closely correlate with lung pathology in experimental infection of mice.

Determination of viral copy number and Mus musculus MX dynamin-like GTPase 1 (Mx1) in the mouse lung was done using quantitative real-time PCR (qRT-PCR). Total RNA was extracted using a ToTALLY RNA kit (catalog number AM1910; Ambion, Austin, Tex., USA). RNA samples were quantified by a NanoDrop spectrophotometer and quality was analyzed on an RNA Nano-drop by the Agilent 2100 bioanalyzer (Agilent Technologies). Synthesis of cDNA was performed with 1 μg of total RNA in a 20 μL reaction mixture by using TaqMan Reverse Transcription Reagents kit from ABI (catalog number N8080234; Applied Biosystems). Amplification was done using 1 μL of cDNA in a total volume of 25 μL using the Faststart Universal SYBR green Master Mix (Roche Applied Science #04913850001). The RSV N-specific reverse transcriptase (RT) primer contained a tag sequence from the bacterial chloramphenicol resistance (Cmr) gene to generate the cDNA, because of self-priming exhibited by RSV RNA. To detect the RSV genome (−) strand, RSV N RT primer 5′-CTGCGATGAGTGGCAGGCACTACAGTGTATTAGACTTRACAGCAGAAG-3′ was used. For PCR assays, we used RSV tag primer CTGCGATGAGTGGCAGGC and primer RSV P GCATCTTCTCCATGRAATTCAGG. To detect Mx1, the mRNA sequence reported under GenBank accession number NM_010846 was used to design amplification primers for qRT-PCR assays. 18S RNA was used as a housekeeping gene for normalization. PCR assays were run in the ABI Prism 7500 Sequence Detection System. Triplicate cycle threshold (C_{T}) values were analyzed in Microsoft Excel by the comparative C_{T} (ΔΔC_{T}) method according to the manufacturer's instructions (Applied Biosystems). The amount of target (2^{-ΔΔCT}) was obtained by normalization to the endogenous reference (18S) sample. RNA isolation, primer design, and qRT-PCR assays were performed at the Molecular Genomics Core, UTMB, Galveston.

### Airway Obstruction

Airway obstruction was measured in unrestrained mice at days one and five post-infection (p.i.) using whole-body barometric plethysmography (Buxco Electronics, Troy, N.Y., USA) to record enhanced pause (Penh)., as previously described (Ivancuic, T., et al., Am J Respir Cell Mol Biol Vol 55, Iss 5, pp 684-696, November 2016). Penh is a dimensionless value that represents a function of the ratio of peak expiratory flow to peak inspiratory flow and a function of the timing of expiration. To establish baseline airway obstruction values, mice were acclimatized to the chambers for five minutes, and respiratory activity was recorded for five minutes. This protocol was designed by Buxco Electronics, and the laboratory staff was trained by the company on the use of this protocol.

### Bronchoaveolar Lavage (Bal)

At days one or five p.i., mice were euthanized with an IP injection of ketamine and xylazine followed by exsanguination via the femoral vessels. An incision was made in the trachea, through which the lungs were flushed twice with 1 mL of cold sterile PBS to obtain BAL fluid (BALF). The chest cavity then was opened for lung collection. Total cell counts were determined by trypan blue staining, followed by counting of viable cells using a hemocytometer. Additionally, 100 μL of BALF was spun onto glass cytocentrifuge slides and stained with H&E (Hema 3 stain, Fisher Scientific) for differential cell counts. The remaining BALF was centrifuged and supernatants were collected, and stored at −80 deg C. until needed for further assays.

### Measurement of Cytokines, Chemokines, Interferon, Total Protein and Elastase

Cytokines, chemokines, interferons, and elastase were all measured using BALF collected at day one p.i. Total proteins were measured using BALF collected at days one and five p.i. Levels of cytokines and chemokines in the BALF were determined with a Bio-Plex Pro Mouse Group I 23-plex panel (BioRad Laboratories, Hercules, Calif., USA). Interferon (IFN)-α and IFN-β were measured by ELISA, following the manufacturer's protocol (PBL Biomedical Laboratories, Piscataway, N.J., USA). Total protein concentrations were determined using the Bradford method (BioRad Laboratories, Hercules, Calif., USA). Neutrophil elastase was measured using a neutrophil elastase ELISA kit (R&D Systems, Minneapolis, Minn., USA). Absorbance for all microplate assays was measured on a SpectraMax 190 microplate reader (MDS Analytical Technologies, Sunnydale, Calif., USA).

### Neutrophil Depletion in Balb/c Mice

In a separate set of experiments, 10 to 12-week-old female BALB/c mice were depleted of neutrophils with an IP injection of 200 μg anti-Ly6G (Clone 1A8; Bio X Cell, West Lebanon, N.H., USA) in a final volume of 100 μL, 12 h prior to infection. Anti-Ly6G was diluted in PBS immediately before being administered to mice. Control mice received the same volume of PBS via IP injection 12 h prior to infection. For infection, mice were intranasal inoculated with 100 μL RSV diluted in PBS at a dose of 5×10⁶ PFU or RSV treated with AgNPs (2 mg/kg or 4 mg/kg) diluted in PBS. Lungs were collected at day five p.i. for determination of viral titer CPE plaque assay using lung homogenate and by qRT-PCR.

### Statistical Analysis

The data were analyzed by a one-way ANOVA followed by Tukey's post hoc test for samples with unequal variances (GraphPad Prism 7; GraphPad Software, Inc., San Diego, Calif., USA). Results are expressed as mean±SEM for each experimental group unless stated otherwise, and p≤0.05 value was selected to indicate significance.

### Results

### AgNPs Reduce RSV Replication in Epithelial Cell Lines

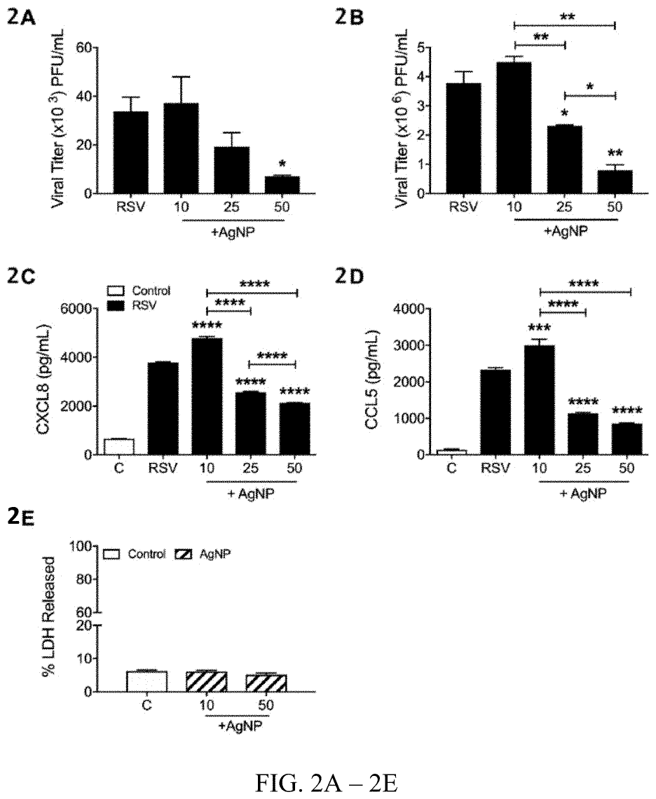

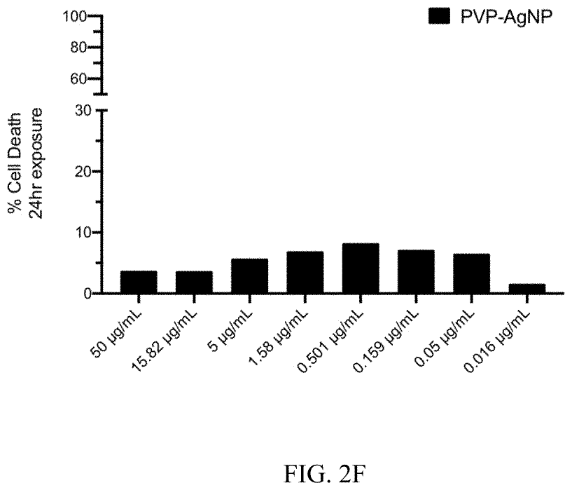

The effect of AgNPs on RSV infection was assessed in vitro by plaque assay in two epithelial cell lines, A549 (MOI: 1; FIG. 2A) and HEp-2 (MOI: 0.01; FIG. 2B). Following incubation of RSV with AgNPs (0, 10, 25, and 50m/mL), both cell lines demonstrated significant dose-dependent reductions in RSV replication. The dose of 50m/mL AgNP was the most effective in either cell type with a decrease of RSV replication by 79% in A549 cells and 78% in HEp-2 cells. This decrease was associated with a significant reduction in CXCL8 (FIG. 2C) and CCL5 (FIG. 2D) secretion by RSV-infected cells. Interestingly, the lowest dose of AgNP (10 μg/mL) induced a small increase in viral replication, coupled with increase secretion of CXCL8 and CCL5 (FIG. 2A-D). This represents a concentration as low as 10 ppm of the AgNPs.

In addition to viral replication, the potential toxicity of AgNPs on A549 cells was also assessed using LDH as an index of cellular damage. There were no notable cytotoxic effects following 24 h of exposure to either the lowest or highest dose of AgNPs (10 or 50 μg/mL) (FIG. 2E).

Additional toxicity testing of AgNPs on epithelial cells was assessed in vitro with a A549 cell line at varying doses of AgNPs (0.016, 0.05, 0.159, 0.501, 1.58, 5, 15.82, and 50m/mL), as shown in FIG. 2F. There were no notable cytotoxic effects following 24 h of exposure to either the lowest or highest dose of AgNPs (0.016 or 50m/mL).

### AgNPs Reduces RSV Replication in the Lung Tissue of Experimentally Infected Mice

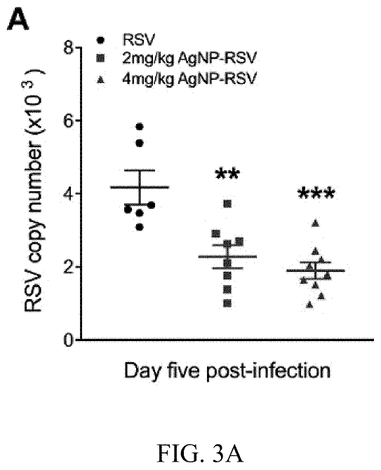

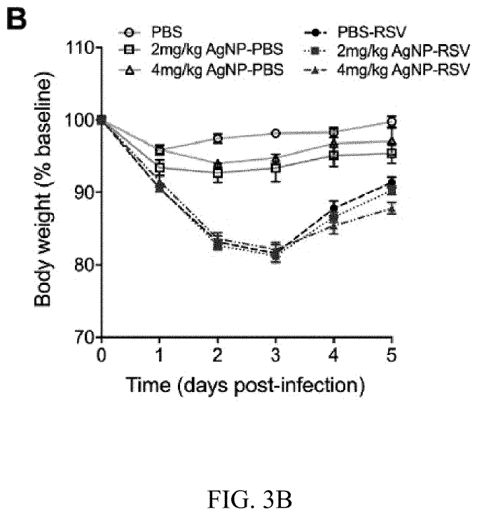

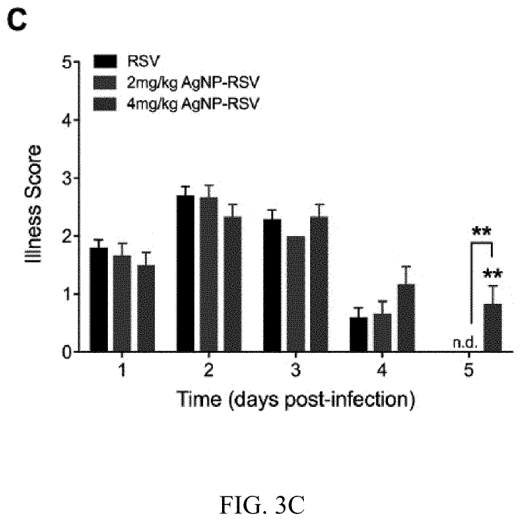

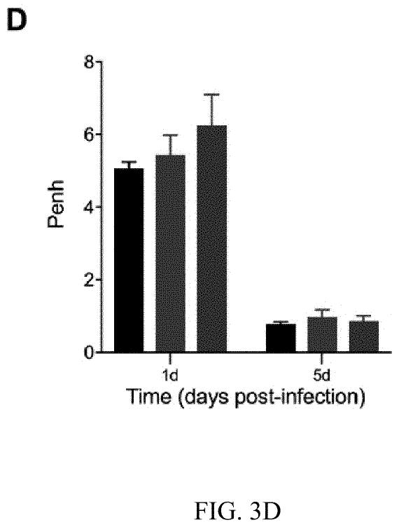

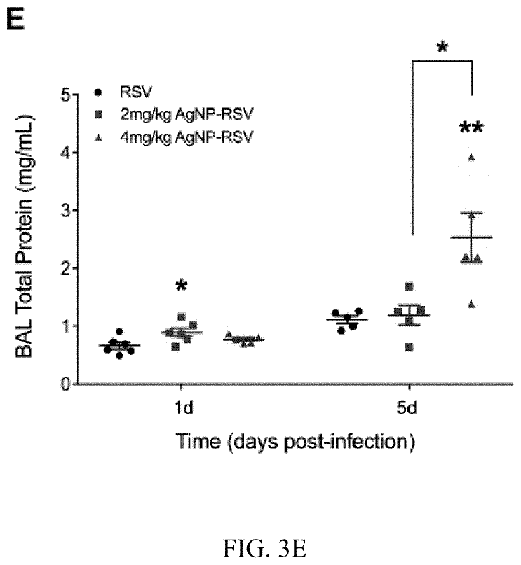

To understand the role of AgNPs in the context of RSV infection in vivo, BALB/c mice were intranasally inoculated with PBS, AgNP-PBS (2 mg/kg or 4 mg/kg), RSV (5×10⁶ PFU), or AgNP-RSV. Lung tissue was collected at day five p.i. to evaluate RSV copy number by qRT-PCR. Mice treated with AgNP-RSV had significant reductions in RSV copy number as compared to the RSV untreated mice (FIG. 3A). Of the two AgNP doses, 4 mg/kg AgNP was most significant, with a decrease of 55%. The dose of 2 mg/kg AgNP elicited a reduction of 45%.

Over the course of the disease, mice were monitored daily for changes in clinical parameters (i.e., bodyweight loss and illness score). Mice inoculated with AgNP-PBS did not display any signs of disease or weight loss over the five-day monitoring period, indicating that AgNPs per se do not lead to clinical illness in mice. Mice inoculated with either dose of AgNP-RSV did not differ in bodyweight loss as compared to RSV untreated mice (FIG. 3B). Mice inoculated with 4 mg/kg AgNP-RSV demonstrated a minor increase in illness score only at day five p.i. (FIG. 3C). No other group had any significant changes in illness score over the five-day period.

Next, to assess the effects of AgNPs on pulmonary function, airway obstruction was analyzed by whole-body plethysmography (Buxco Electronics, Inc., Sharon, Conn., USA) and expressed as enhanced pause (Penh). Mice inoculated with either dose of AgNP-PBS had no notable changes to baseline Penh at day one or day five p.i. (data not shown). Mice inoculated with AgNP-RSV showed an increasing trend in baseline Penh values, but were statistically similar to the RSV untreated mice at both time points (FIG. 3D). We also evaluated the concentration of total protein as a marker of epithelial damage and increased vascular permeability. At day 1 p.i., mice inoculated with 2 mg/kg AgNP-RSV demonstrated an average value of 0.89 mg/mL, which was a marginal increase compared to the RSV untreated group that demonstrated total protein levels of 0.66 mg/mL (FIG. 3E). Mice inoculated with 4 mg/kg AgNP-RSV had total protein levels comparable to the RSV untreated mice. At day 5 p.i., mice inoculated with 4 mg/kg AgNP-RSV demonstrated a significant increase in total protein concentration with a value of 2.53 mg/mL as compared to 1.11 mg/mL for RSV untreated mice. Mice inoculated with 2 mg/kg AgNP-RSV had, total protein levels comparable to the RSV untreated mice (FIG. 3E).

AgNPs Decrease Many RSV-Induced Cytokines and Chemokines, while Increasing Those Associated with Neutrophil Recruitment and Activation

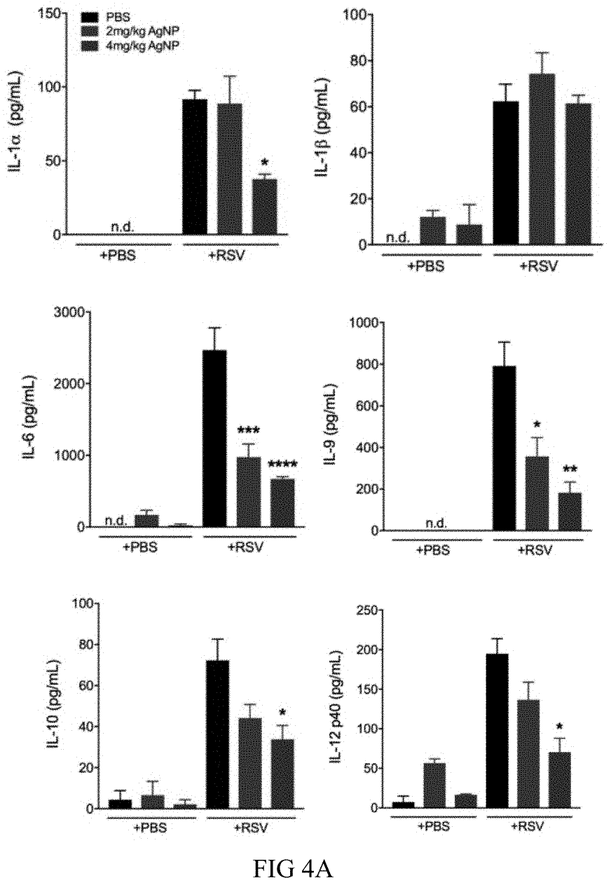

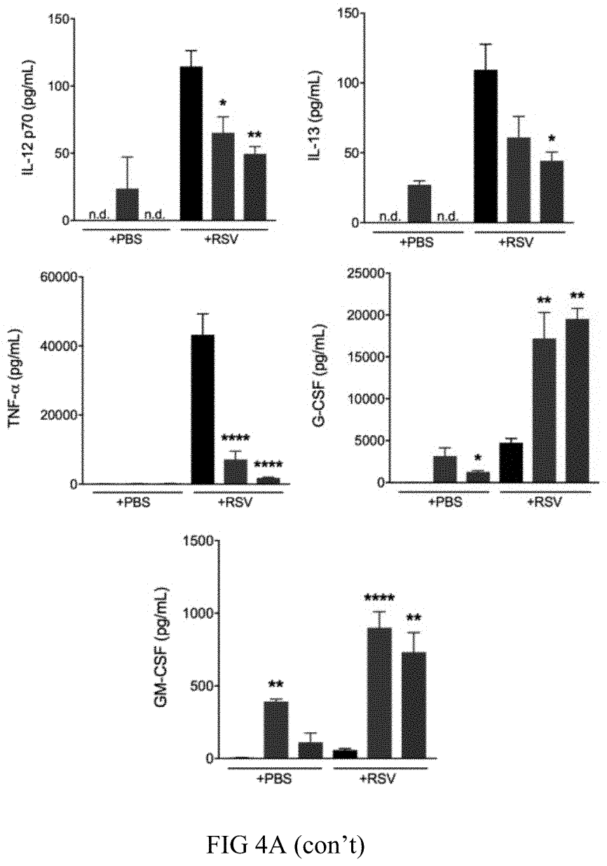

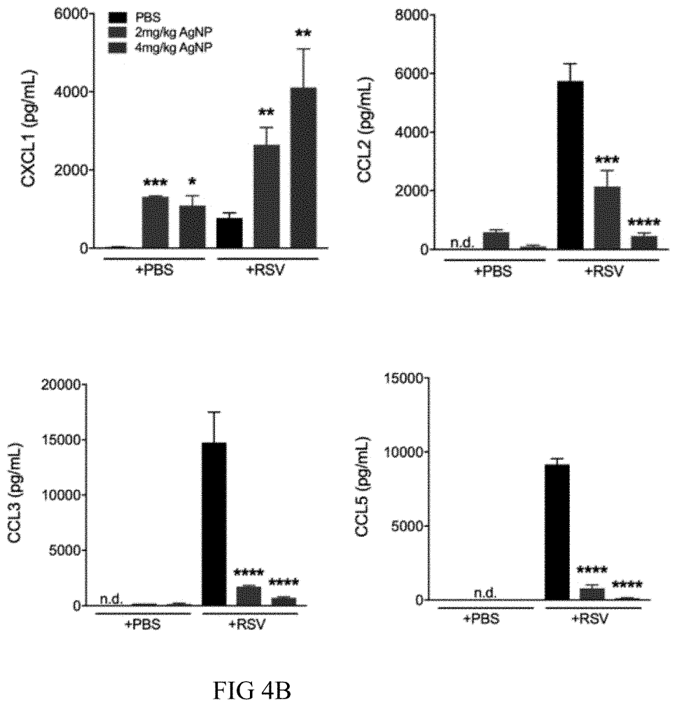

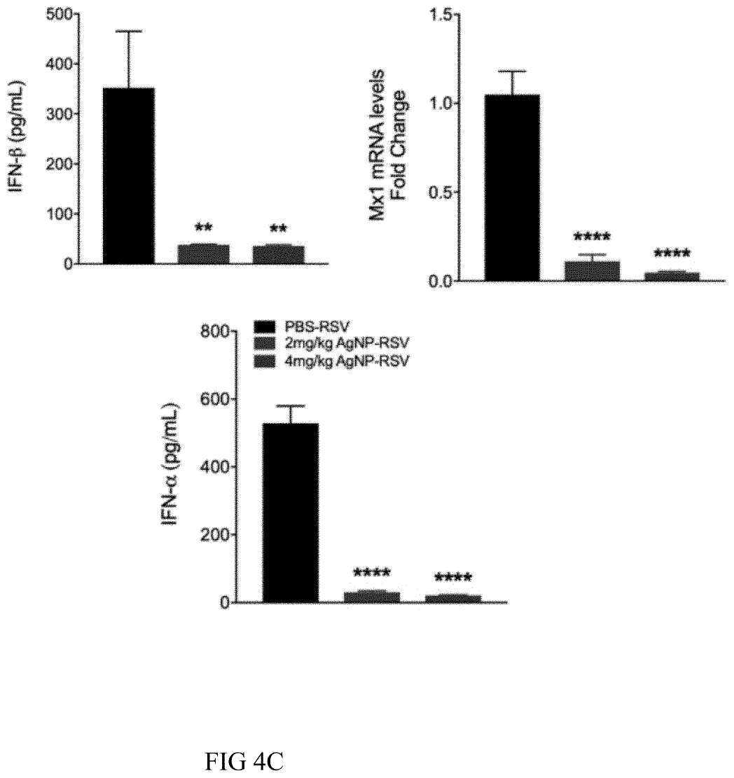

To investigate the immunomodulatory effects of AgNPs during RSV infection, cytokine and chemokine concentrations of the BALF at day one p.i. were evaluated by a multiplex cytokine array. In mice inoculated with AgNPs, regardless of infection status, G-CSF and GM-CSF were significantly increased (FIG. 3A). Conversely, inflammatory and immunomodulatory cytokines, such as interleukin (IL)-1α, IL-6, IL-9, IL-10, IL-12p40, IL-12-p70, IL-13, and TNF-α were significantly decreased in mice inoculated with either dose of AgNP-RSV (FIG. 4A). In addition to G-CSF and GM-CSF, the chemokine CXCL1 (KC) was significantly increased in all mice inoculated with AgNPs, regardless of infection status. Chemokines associated with viral replication, such as CCL3 (MIP-1α) and CCL5 (RANTES), were significantly decreased (FIG. 4B).

To assess type-I IFN production, an ELISA (PBL Biomedical Laboratories, Piscataway, N.J., USA) was conducted at day one p.i. using the BALF of mice inoculated with AgNP-RSV and compared with RSV untreated mice. This was further supported by testing for the Mx1 gene within the lung tissue by qRT-PCR. IFN-α, IFN-β, and Mx1 were all significantly decreased following inoculation with either dose of AgNP-RSV as compared to the RSV untreated mice (FIG. 4C).

### AgNPs Increase and Maintain Neutrophil Cell Counts in the BALF, Regardless of Infection Status

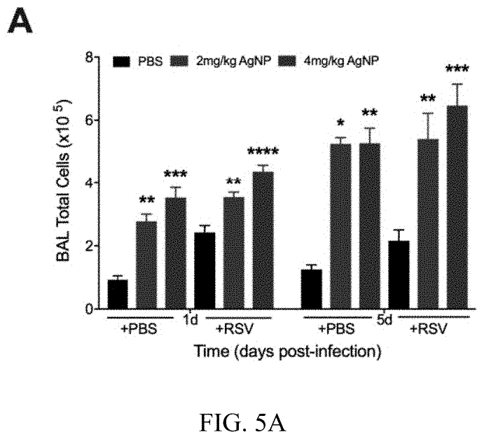

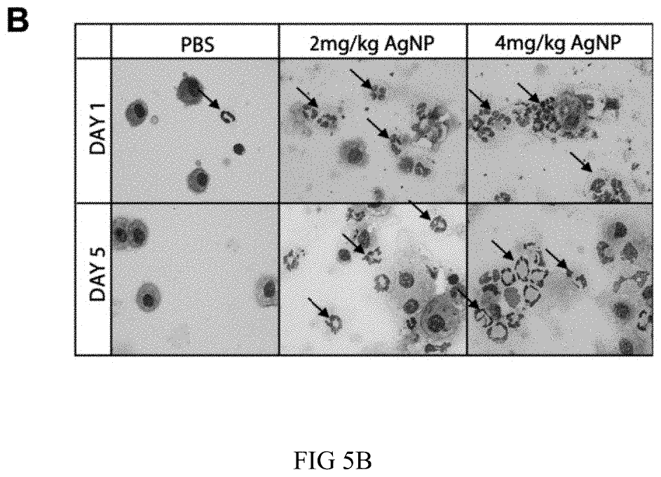

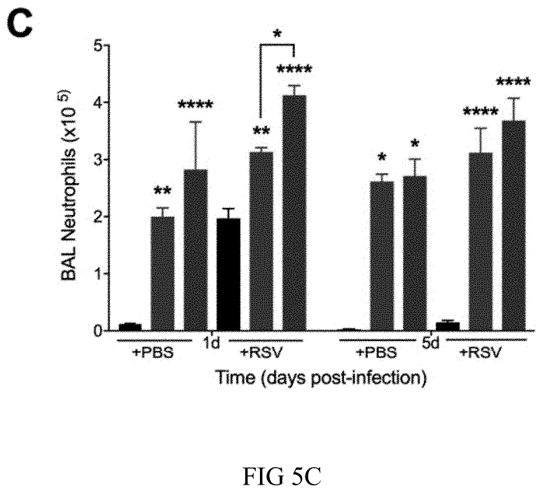

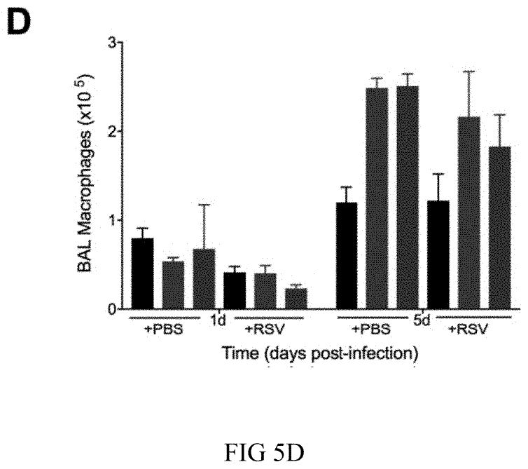

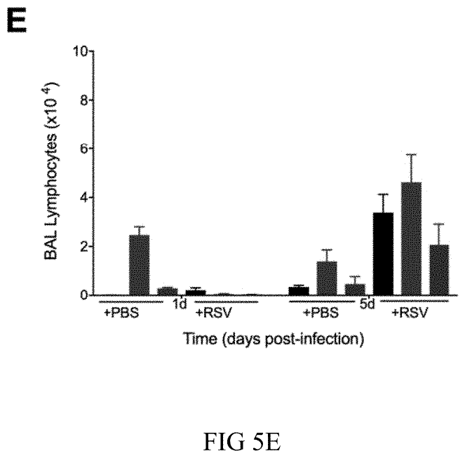

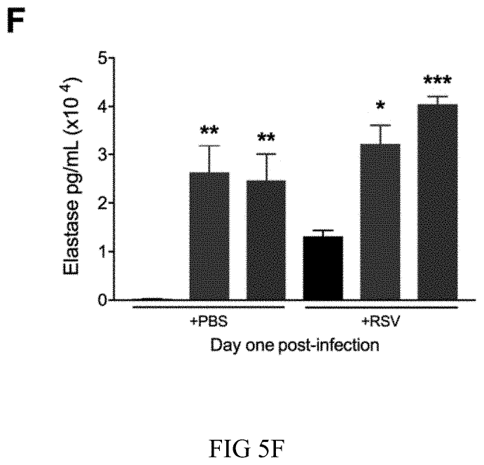

To determine whether AgNP treatment affected the cellular composition, BAL samples were collected from inoculated mice at days one and five p.i. for total and differential cell counts. The total cell count was significantly greater in the BALF of all AgNP inoculated mice at days one and five, regardless of infection status (FIG. 5A). Differential cell counts revealed this increase to be primarily due to a significant increase in the number of neutrophils at both time points (FIG. 5B,C). Macrophage cell counts were unaffected in either group at day one p.i. and had an increasing trend in cell count at day five p.i., regardless of infection status (FIG. 5D). Lymphocyte cell counts were comparable to the respective controls at both days one and five p.i. (FIG. 5E).

Next, we wanted to understand if the neutrophils present in the BALF of mice inoculated with either dose of AgNPs were activated using elastase as a marker of neutrophil activation. BALF collected at day one p.i. demonstrated significant increases in elastase in all AgNP inoculated mice, regardless of infection status. A dose-dependent increase in elastase production was observed in AgNP-RSV treated mice as compared to RSV untreated mice (FIG. 5F).

### Neutrophils are a Primary Mechanism of the Antiviral Activity by AgNPs In RSV-Experimentally Infected Mice

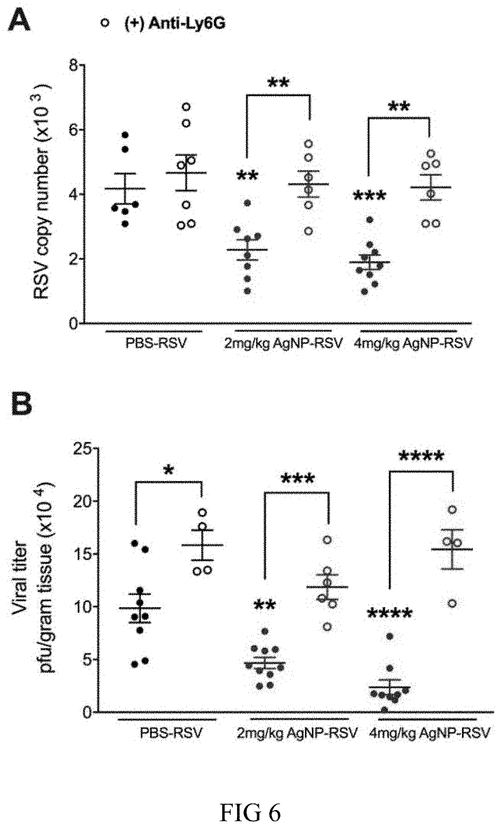

Some studies have shown AgNPs to increase neutrophil cell counts within the BALF of treated mice, but few have investigated their activity following recruitment (Haberl, N, et al. Beilstein J. Nanotechnol. 2013, 4, 933-940; Luo, Y. et al. Bio Med Res. Int., 2015). Neutrophils have also been suggested to have antiviral capabilities during RSV infection, leading us to evaluate the function of neutrophils in AgNP-RSV treated mice. Mice were depleted of neutrophils with an injection of anti-Ly6G clone 1A8 12 h prior to inoculation with RSV (5×10⁶ PFU), or AgNP-RSV. In neutrophil-depleted mice inoculated with 2 mg/kg AgNP-RSV or 4 mg/kg AgNP-RSV, the viral copy number at day five p.i. was found to be similar to the viral copy number in neutrophil-depleted RSV untreated mice, indicating a reversal of the antiviral effect noted in immunocompetent mice (FIG. 6A). This was further supported by a viral plaque assay using the lung homogenate of infected mice (FIG. 6B). These results support neutrophils as a primary mechanism of the antiviral activity generated by AgNPs against experimental RSV infection in mice.

### Discussion of Results

A significant dose-dependent reduction of RSV replication in both HEp-2 and A549 cell lines was reported, with the strongest antiviral effect elicited by a dose of 50 μg/mL AgNP. In addition, cells exposed to this dose of AgNPs were found to release levels of LDH similar to that of the control, demonstrating that AgNPs at the dose of 50 μg/mL are not toxic to epithelial cells. The effectiveness of 50 μg/mL AgNPs in epithelial cell lines is in agreement with work previously reported examining the effects of AgNPs on influenza strains H1N1 and H3N2. To confirm the findings in vivo, BALB/c mice were inoculated with AgNP-RSV, and lung tissue was collected at day five p.i. to evaluate viral titer. Inoculation with AgNP-RSV resulted in significant reductions in viral titer as compared to RSV untreated mice. This demonstrates, for the first time, the effectiveness of AgNPs against experimental RSV infection in mice.

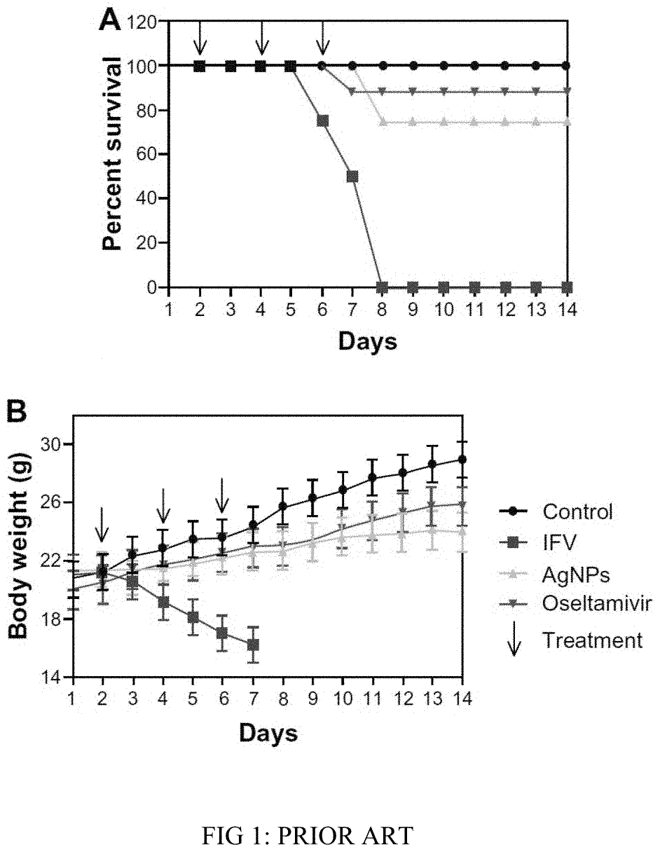

The mechanism to the antiviral effect against RSV in epithelial cell lines is likely due to be the attachment of AgNPs to surface glycoproteins. By doing so, AgNPs would interfere with RSV's ability to initiate attachment with the proper receptors, preventing fusion of the virus to the host cell. This would leave RSV in the extracellular space where it is unable to propagate, resulting in the reduction of syncytia formation seen in the plaque assays. The basis of this mechanism has been investigated with other RNA viruses, such as human immunodeficiency virus type-1 (HIV-1). AgNPs were shown to directly associate with the surface glycoprotein gp120, preventing HIV-1 from interacting with host receptors. The gp120 glycoprotein has also been found to have some structural similarities with the RSV-F surface glycoprotein, supporting the hypothesis of a direct association of AgNPs with RSV. Interestingly, exposure of 10m/mL AgNP to RSV in A549 and HEp-2 cell lines resulted in a slight increase in RSV replication and secretion of CXCL8 and CCL5. Though it is not clear at the present time why a low dose of AgNPs would have such an impact on viral parameters, we believe that insufficient coating of the virus with AgNPs would allow the virus to continue infecting epithelial cells. This enhancement of replication was not appreciated with increasing doses of AgNPs, suggesting that RSV virions are more efficiently coated with higher doses of AgNPs (FIG. 1A-B).

Although a potential direct antiviral mechanism has been previously described, cell culture lacks the interaction of a complex immune system. Therefore, to elucidate potential antiviral mechanisms due to AgNPs in vivo, investigation of the cytokine, and cellular composition of the BALF collected from inoculated mice was undertaken. AgNP-RSV infected mice were found to have significant reductions in many pro-inflammatory and viral associated markers, such as IL-6, TNF-α, CCL5 (RANTES), and type I IFNs. Additionally, significant reductions were also noted with IL-1α, IL-9, IL-10, IL-12p40, IL-12p70, IL-13, CCL2 (MCP-1), and CCL3 (MIP-1α). Previous data in the field of RSV has suggested that a downregulation in pro-inflammatory cytokines, such as TNF-α corresponds with significantly improved clinical disease (i.e., bodyweight loss, illness score, airway obstruction). Conversely, the study demonstrated no significant changes to clinical parameters in AgNP-RSV inoculated mice, despite significant decreases in an array of RSV-induced cytokines. Upregulations in cytokines and chemokines responsible for neutrophil recruitment and/or activation (i.e., elastase, CXCL1, G-CSF, and GM-CSF) have been linked to heightened airway inflammation and RSV bronchiolitis. As shown in FIG. 3, CXCL1, G-CSF, and GM-CSF were all found to be significantly increased following AgNP inoculation. Therefore, the beneficial effects typically associated with reductions in pro-inflammatory cytokines are likely counteracted by the strong presence of these neutrophil associated cytokines.

Consistent with increases in cytokines associated with neutrophil recruitment, the number of neutrophils in the BALF was also found to be significantly increased. These findings are in agreement with other reports demonstrating an upregulation of neutrophils in many rodent models following inoculation with AgNPs. The mechanism to this neutrophilia is believed to be an initial stimulation of macrophages to release CXCL1 (KC), but the role of neutrophils during AgNP exposure is still poorly understood. RSV is also known to induce neutrophilia during the early stages of the disease, but the activity of neutrophils during RSV infection has only recently begun to be properly elucidated, and reports remain contradictory. Therefore, to evaluate the role of neutrophils following AgNP-RSV exposure, we depleted mice of neutrophils using an injection of anti-Ly6G. AgNP-RSV treated mice that had been depleted of neutrophils were found to have viral titers similar to RSV untreated neutrophil-depleted mice, demonstrating a reversal of the antiviral effect observed in AgNP-RSV neutrophil immunocompetent mice. These findings suggest AgNPs modulate the function of neutrophils in RSV infection to enhance their antiviral activity.

Toxicity studies of AgNPs in the lung have been performed in a variety of rodent models and have shown overall that AgNPs induce minor airway mucosa thickening and cellular infiltration (primarily neutrophils), but generate no major alterations to lung function, even following 28 days of continuous exposure. This is consistent with our findings, which demonstrate a sizable neutrophil influx to the BAL fluid through day five in mice inoculated with PBS-AgNPs, but without evidence of clinical disease (i.e., bodyweight-loss, illness score, airway obstruction). Similarly, although both doses of AgNPs resulted in significantly increased neutrophil recruitment/activation in RSV-infected mice, clinical parameters were comparable to those observed in RSV-infected untreated mice at the peak of disease (day 2-4). At the time of recovery (day 5), we observed some delay in the regaining of bodyweight and clinical disease along with higher total protein content in the BAL only in the group of infected mice that had received the higher dose of AgNPs (4 mg/kg), a result of the higher number of activated neutrophils in the BALF (FIG. 2) as previously suggested. However, mice that received the 4 mg/kg AgNP dose also exhibited the largest reduction in RSV lung titers, as well as in all pro-inflammatory cytokines (i.e., TNF-α, IL-6), suggesting that AgNPs potentiate the neutrophil anti-RSV activity as their major antiviral function in the experimental mouse model.

In conclusion, the study demonstrates that AgNPs effectively reduce RSV replication and production of pro-inflammatory cytokines in epithelial cell lines and in mouse lung. In the mouse model, the antiviral activity appears to be mediated to a large extent by neutrophils, which are recruited in higher number to the airways and activated via a neutrophil-specific program of cytokines that include CXCL1, G-CSF, and GM-CSF. These findings contribute to the understanding of AgNP bioactivity in the lung while providing insights on the role that neutrophils play in the host response against infections caused by RSV.

This application incorporates all cited references, patents, and patent applications by references in their entirety for all purposes.

What is claimed is:

1. A composition for inhibiting respiratory viruses including RSV and SARS-CoV2 related infections comprising; encapsulated metal oxides or chelated metal oxide molecules wherein said metal oxide(s) molecules are silver oxide molecules in solution and wherein said silver oxide(s) molecules are present in a concentration of between 5 and 250 parts per million in said solution.

2. The composition of claim 1, wherein a solvent provides a liquid solution of encapsulated metal oxides or chelated metal oxide molecules wherein said solvent is selected from one or more of a group consisting of water, alcohol, plant-derived glycerine, and aloe.

3. The composition of claim 1, wherein said silver oxide molecules are encapsulated with one or more biopolymers that have a core and one or more shells that enclose said core.

4. The composition of claim 2, wherein said shells are comprised of a nonlamellar amorphous material and wherein said core contains said silver oxide molecules.

5. The composition of claim 2, wherein said biopolymers are polyvinylpyrrolidone (PVP) as provided in Structure (1), or another water-soluble polymer of N-vinylpyrrolidone (NVP) or a derivative thereof as provided in structure (2).

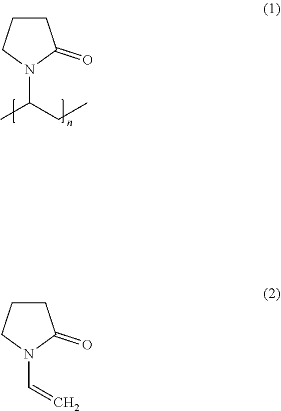

6. The composition of claim 4, wherein said silver oxide molecule encapsulated with said biopolymers are either silver oxide or chelated silver oxide nanoparticles with a particle size of between 1 nm and 20 nm.

7. The composition of claim 6, wherein said silver oxide nanoparticles exhibit a high degree of inhibition of said viruses specifically in a range of 8 nm to 11 nm.

8. The composition of claim 1, wherein said silver oxide nanoparticles reduce RSV replication and production of pro-inflammatory cytokines in epithelial cell lines and in mouse lung that is mediated by neutrophils.

9. A method of inhibiting respiratory viruses including RSV and SARS-CoV2 related infections comprising inhalation and/or intranasal administration of doses of encapsulated metal oxide or chelated metal oxide molecules wherein said metal oxides are silver oxides and are provided in a liquid solution wherein a solvent provides a solution of encapsulated metal oxides or chelated metal oxide molecules wherein said solvent is selected from one or more of a group consisting of water, alcohol, plant-derived glycerine, and aloe.

10. The method of claim 9, wherein said method includes parenteral administration of said molecules.

11. The method of claim 9, wherein said method is useful in reducing RSV mRNA levels, RSV protein levels and RSV viral titers in a subject, said subject including a mammal, and a human.

12. The method of claim 9, wherein administration of multiple doses of encapsulated silver oxide or chelated silver oxide molecules over a course of days provides a therapeutically effective amount of chelated silver oxide.

13. The method of claim 9, wherein a first of a plurality of doses is administered in a prophylactic manner before a subject is infected with RSV or a SARS-CoV2 virus related infection, such as COVID-19.

14. The method of claim 9, wherein an additional risk reduction benefit of an inhalation treatment of an encapsulated metal oxide or chelated metal oxide composition for ventilated patients includes suppression of biofilm formation inside an endotracheal or tracheostomy tube.

15. The method of claim 9, wherein said liquid solution is formulated to have an osmolality ranging from 200-400 mOsm/kg.

16. The method of claim 9, wherein said liquid solution is a buffered solution and wherein a pH of said liquid solution is between 5 and 8.

17. The method of claim 98, wherein a composition is administered as an aerosolized liquid such as a nasal spray.

18. The method of claim 9, wherein said aerosolized liquid is produced by a nebulizer.

19. The method of claim 9, wherein 0.1 ml to 0.6 ml of said aerosolized liquid comprising an encapsulated metal oxide or metal oxide chelate is administered to each nostril.

20. The method of claim 9, wherein a plurality of doses can be administered daily, wherein a plurality includes two, three, four, or five doses.

21. The method of claim 9, wherein administering of said plurality of doses reduces RSV protein, mRNA, or titer in a cell of a human or mammalian respiratory tract to at least a level of administering a single dose that equals a dose provided by said plurality of doses.

22. The method of claim 9 wherein administering of said plurality of doses by inhalation delivers a total dose of between 0.1 and 0.6 mg/kg of an encapsulated metal oxide or metal oxide chelate to a mammal and/or human.
